(19) United States
(12) Patent Application Publication
(10) Pub. No.: US 2008/0145412 A1
(43) Pub. Date: Jun. 19, 2008

(54) METHOD OF PROMOTING ANGIOGENESIS

(75) Inventors: Catherine Middleton-Hardie, Hayward, CA (US); Mirella Lazarov, Hayward, CA (US); David Rosen, Hayward, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Acologix, Inc.

(21) Appl. No.: 11/735,727

(22) Filed: Apr. 16, 2007

Related U.S. Application Data
(60) Provisional application No. 60/792,744, filed on Apr. 17, 2006.

(51) Int. Cl. A61K 9/70; A61K 38/02; A61K 38/10; A61K 38/16

(52) U.S. Cl. 424/447; 514/2; 514/13; 514/12

(57) ABSTRACT

The present invention is a method for promoting angiogenesis by administering a formulation comprising an effective amount of a peptidic compound, e.g., a peptide having the sequence TDLQERGDNDISPFSGDGQPFKD (SEQ ID NO: 30), which enhances angiogenesis through the induction of pro-angiogenic factors such as FGF2, TGFβ and fibronectin. Formulations may be administered by catheter to treat or prevent diseases and conditions which involve angiogenesis by administering an effective amount of the peptidic compound alone or in combination with one or more additional therapeutic agents.

### CROSS REFERENCES

This application claims the benefit of U.S. Provisional Application No. 60/792,744, filed April 17, 2006, which application is incorporated herein by reference.

### BACKGROUND

Angiogenesis is a natural growth and development process in which new blood vessels are formed from extant capillaries. The regulation of angiogenesis plays an important role in bone formation, embryonic development, inflammation, wound healing, and contribute to pathological conditions such as tumor growth, diabetic retinopathy, rheumatoid arthritis, and chronic inflammatory diseases.

Angiogenesis involves the proliferation of endothelial cells which line the walls of blood vessels. Not only does angiogenesis increase endothelial cell proliferation but also comprises a cascade of additional events, including protease secretion by endothelial cells, degradation of the basement membrane, migration through the surrounding matrix, proliferation, alignment, differentiation into tube-like structures, and synthesis of a new basement membrane.

Several angiogenic agents with different properties and mechanisms of action are well known in the art. For example, acidic and basic fibroblast growth factor (FGF), transforming growth factor alpha (TGF-α) and beta (TGF-β), tumor necrosis factor (TNF), platelet-derived growth factor (PDGF), vascular endothelial cell growth factor (VEGF), and angiogenin are potent and well-characterized angiogenesis-promoting agents. In addition, both nitric oxide and prostaglandin (a prostacyclin agonist) have been shown to be mediators of various angiogenic growth factors, such as VEGF and bFGF. However, the therapeutic applicability of some of these compounds, especially as systemic agents, is limited by their potent pleiotropic effects on various cell types.

The promotion of angiogenesis has recently been of particular interest for therapeutic purposes. Stimulation of angiogenesis can aid in bone and tissue regeneration, wound healing, the vascularizing of skin grafts, and the enhancement of collateral circulation where there has been vascular occlusion or stenosis (e.g., to develop a “biobypass” around an obstruction due to coronary, carotid, or peripheral arterial occlusion disease). There is an intense interest in factors that are well-tolerated by the subject, but that are of high potency in effecting stimulation of angiogenesis.

Clearly, there is a significant demand for a therapeutic agent that is well-tolerated by the subject but is of high potency in effecting stimulation of angiogenesis.

### SUMMARY

The present invention is a method for promoting angiogenesis by administering a formulation comprising an effective amount of a peptidic compound, e.g. a peptide having the sequence TDLQERGDNDISPFSGDGQPFKD (SEQ ID NO: 30), which enhances angiogenesis through the induction of pro-angiogenic factors such as FGF2. TGFβ and fibronectin. The methods of the present invention may be used to treat or prevent diseases and conditions which involve angiogenesis by administering an effective amount of the peptidic compound alone or in combination with one or more additional therapeutic agents.

An aspect of the invention involves a method of promoting angiogenesis in a patient whereby a formulation comprised of a carrier and a peptide of the invention is brought into contact with the target cells for a sufficient period of time and in a sufficient amount to stimulate angiogenesis.

An aspect of the invention for promoting angiogensis is that the administering of the formulation is carried out by a route chosen from intramuscular, inhalation, topical, transdermal, intravenous, intra-arterial and intra-pericardial.

In accordance with the invention a formulation of the invention can be administered locally or systemically and can be administered in connection with surgery in order to aid in healing any type of surgical wound.

In accordance with one embodiment of the invention the formulation of the invention is administered using a catheter which catheter can be a balloon catheter which can be used by placing a peptide of the invention on an exterior surface of the balloon and bringing the balloon in contact with an interior surface of a blood vessel so that the peptide promotes angiogenesis in the endothelial lining of the blood vessel.

Another aspect of the invention is a bandage which bandage is comprised of a structural base material which may include a fabric such as cotton or other natural or synthetic materials or a gel. A base material has thereon or infused, dispersed or dissolved therein a peptide or peptide formulation of the invention.

These and other aspects, objects, advantages, and features of the invention will become apparent to those persons skilled in the art upon reading the details of the subject invention, as more fully described below.

### BRIEF DESCRIPTIONS OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

Included in the drawings are the following figures:

FIG. 1 depicts data demonstrating that AC-100 is able to increase the production of FGF-2 in bone. AC-100 at both 1 and 10 uM concentrations increased the production of FGF-2 in mouse calvaria organ cultures. Increases were found in both tissue lysates and in the conditioned media of cultures treated with AC-100.

FIGS. 2A-2D depict data demonstrating that the production of FGF-2 was increased over a several day time period ranging from approximately 1 hr to 5 days (FIG. 2A). In addition, this response roughly correlated with expression of PGE-2 following AC-100 treatment (2B), TGF-β (2C) and Fibronectin (2D). Increases in PGE2 in the same cultures were observed from days 2-7.

### DETAILED DESCRIPTION OF THE INVENTION

Before the methods, peptides, analogs, and formulations of the present invention are described, it is to be understood that this invention is not limited to any particular embodiment described, as such may, of course, vary. It is also to be understood that the terminology used herein is with the purpose of describing particular embodiments only, and is not intended to limit the scope of the present invention which will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent there is a contradiction between the present disclosure and a publication incorporated by reference.

It must be noted that as used herein and in the appended claims, the singular forms a “and”, and “the” include plural referents unless the context clearly dictates otherwise. Thus, for example, reference to “a peptide” includes a plurality of such peptides and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “peptide” and “peptidic compound” are used interchangeably herein to refer to a polymeric form of amino acids of from about 10 to about 50 amino acids, which can comprise coded and non-coded amino acids, chemically or biochemically modified or derivatized amino acids, L- or D-amino acids, peptides having modified peptide backbones, and peptides comprising amino acid analogs. The peptidic compounds may be polymers of: (a) naturally occurring amino acid residues; (b) non-naturally occurring amino acid residues, e.g. N-substituted glycines, amino acid substitutes, etc.; or (c) both naturally occurring and non-naturally occurring amino acid residues/ substitutes. In other words, the subject peptidic compounds may be peptides or peptoids. Peptoid compounds and methods for their preparation are described in WO 91/19735, the disclosure of which is herein incorporated by reference. A peptide compound of the invention may comprise 23 amino acids or from not less than 18 to not more than 28 amino acids or from not less than 20 to not more than 26 amino acids. The active amino acid sequence of the invention comprises three motifs which may be overlapping which are: an integrin binding motif sequence; a glycosaminoglycan binding motif sequence; and a calcium-binding motif.

The terms “treatment”, “treating” and the like are used herein to generally mean obtaining a desired pharmacologic and/or physiologic effect, e.g., stimulation of angiogenesis. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing a disease or condition (e.g., preventing the loss of a skin graft or a re-attached limb due to inadequate vascularization) from occurring in a subject who may be predisposed to the disease but has not yet been diagnosed as having it; (b) inhibiting the disease, e.g., arresting its development; or (c) relieving the disease (e.g., enhancing the development of a bio-bypass around an obstructed vessel to improve blood flow to an organ). In the context of the present invention, stimulation of angiogenesis is employed for a subject having a disease or condition amenable to treatment by increasing vascularity and increasing blood flow.

“Treating” as used herein covers treating a disease in a vertebrate and particularly a mammal and most particularly a human, and includes:
(a) preventing the disease from occurring in a subject which may be predisposed to the disease but has not yet been diagnosed as having it;
(b) inhibiting the disease, i.e. arresting its development; or
(c) relieving the disease, i.e. causing regression of the disease.

The term “antibody” is meant an immunoglobulin protein capable of binding an antigen. The term “antibody” as used herein is intended to include antibody fragments (e.g. F(ab′)₂, Fab′, and Fab) capable of binding an antigen or antigenic fragment of interest.

The term “binds specifically” is meant high avidity and/or high affinity binding of an antibody to a specific peptide—specifically a peptide of the invention. Antibody binding to its specific target epitope is stronger than the binding of the antibody to other epitopes on the peptide or to other epitopes on other peptides. Antibodies which bind specifically to a peptide of interest may be capable of binding to other peptides at a weak, yet detectable level (e.g. 10% or less of the binding shown to the peptide of interest). Such weak binding or background binding, is readily discernable from the specific antibody binding to the peptide of interest, e.g. by the use of appropriate controls.

The terms “subject,” “individual,” “patient,” and “host” are used interchangeably herein and refer to any vertebrate, particularly any mammal and most particularly including human subjects, farm animals, and mammalian pets.

### Peptidic Compounds

A peptidic compound used in the methods of the present invention is a peptide comprising from not less than 10 to not more than 30 amino acids. The amino acids are preferably one of the twenty naturally occurring L-amino acids. However, D-amino acids may be present as may amino acid analogs. A peptide of the invention will comprise both of an integrin binding motif and a glycosaminoglycan binding motif. Individual amino acids may be present in the peptides in either the L or the D isoform, but preferably in the L form. A peptide of the invention can be amidated or non-amidated on its C-terminus, or carboxylated or non-carboxylated on its N-terminus. A compound of the invention is still further characterized by biological activity i.e. it promotes angiogenesis.

A peptidic compound of the invention exhibits one or more of the following properties when administered in an effective amount to an individual: (1) promotes angiogenesis; (2) has no adverse effects; and (3) accelerates wound healing.

An example of the integrin binding motif in a peptide of the invention is an RGD sequence but not necessarily limited to it. Specific examples of the peptides of the invention which comprise the RGD sequence as the terminal sequence include the following:

| | |
|---|---|
| RGDNDISPFSGDG | (SEQ ID NO:1) |
| RGDNDMSPFSGDG | (SEQ ID NO:2) |
| RGDNDVPPFSGDG | (SEQ ID NO:3) |
| RGDNDISPFSGDG | (SEQ ID NO:4) |
| RGDNDISPFSGDGQPFKD | (SEQ ID NO:5) |
| RGDNDMSPFSGDGQPFKD | (SEQ ID NO:6) |
| RGDNDVPPESGDGQHFMH | (SEQ ID NO:7) |
| RGDNDISPFSGDGQPFKDIPGKG | (SEQ ID NO:8) |
| RGDNDISPFSGDGQPFKDIPGKGEATGPDL | (SEQ ID NO:9) |
| SGDGLQERGD | (SEQ ID NO:10) |
| SGPDLLVRGD | (SEQ ID NO:11) |
| SGDGYTDLQERGD | (SEQ ID NO:12) |
| SGDGPDLLVRGD | (SEQ ID NO:13) |
| IPSDFEGSGYTDLQERGD | (SEQ ID NO:14) |
| SKVKKIPSDFEGSGYTDLQERGD | (SEQ ID NO:15) |
| IDYLKHLSKVKKIPSDFEGSGYTDLQERGD | (SEQ ID NO:16) |

Specific examples of the peptides of the invention which comprise the RGD internally include the following:

| | |
|---|---|
| ERGDNDISPFSGDG | (SEQ ID NO:17) |
| YTDLQERGDNDISPFSGDG | (SEQ ID NO:18) |
| YTDLQERGDNDISPFSGDGQPFKD | (SEQ ID NO:19) |
| SDFEGSGYTDLQERGDNDISPFSGDG | (SEQ ID NO:20) |
| SGYTDLQERGDNDISPF | (SEQ ID NO:21) |
| PDLLVRGDNDVPPFSGDG | (SEQ ID NO:22) |
| PDLLVRGDNDVPPFSGDGQHFMH | (SEQ ID NO:23) |
| TDLQERGDNDMSPFSGDGQPFKD | (SEQ ID NO:24) |
| KKIPSDFEGSGYTDLQERGDNDISPF | (SEQ ID NO:25) |
| YTDLQERGDNDISPFSGDGQPFKDIPGKGE | (SEQ ID NO:26) |
| KKIPSDFEGSGYTDLQERGDNDISPFSGDG | (SEQ ID NO:27) |

A peptide of the invention comprises a glycosaminoglycan-binding motif. A glycosaminoglycan binding motif has the consensus sequence SG. In some embodiments, a glycosaminoglycan binding motif has the sequence SGDG (SEQ ID NO:28). Although not necessarily essential, a peptide of the invention could also comprises a calcium binding motif. For example, in some embodiments, a calcium binding motif has a consensus sequence of DXDXSXFXGXXQ (SEQ ID NO:29), wherein X is any amino acid or amino acid analog.

Calcium binding motifs are known in the art and have been described amply. See, for example, Springer et al. (2000) Cell 102:275-277; Kawasaki and Kretsinger (1995) Protein Prof. 2:305-490; Moncrief et al. (1990 J. Mol. Evol. 30-522-562; Chauvaux et al. (1990) Biochem. J. 265:261-265; Bairoch and Cox (1990) FEBS Lett. 269:454-456; Davis (1990) New Biol. 2:410-419; Schaefer et al. (1995) Genomics 25:638-643; and Economou et al. (1990) EMBO J. 9:349-354. Any known calcium binding motif can be included in a peptide compound of the invention.

A peptide of the invention may comprise both an integrin binding motif and a glycosaminoglycan binding motif. These motifs may be present in the peptide in any order relative to one another. The motifs may be separated from one another by one, two, three, four, five, six, seven, eight, nine, or ten amino acids, or more. As one non-limiting example, the invention includes a peptide having the sequence TDLQERGDNDISPFSGDGQPFKD (SEQ ID NO: 30). This peptide is referred here as AC-100.

All or any of the amino acids in the above sequences may be in the D- or L-conformation and may be substituted with equivalent analogs. The preferred embodiments comprise naturally occurring amino acids in the L-conformation.

All or any of the above sequences may be amidated, non-amidated, or otherwise modified on their C-terminus, or carboxylated, non-carboxylated, or otherwise modified on their N-terminus.

In addition, multimers of any of the foregoing peptides are provided. Multimers include dimers, trimers, tetramers, pentamers, hexamers, etc. Thus, a peptide of the invention having a length of from about 10 to about 30 amino acids can be multimerized, optionally with an intervening linker, such that a subject peptide occurs in tandem arrays of two, three, four, five, six, or more copies. Furthermore, two or more different peptides of the invention can be multimerized with one another, forming “heteromultimers.” Thus, e.g., a multimer may comprise a first and a second peptide, linked together by peptide bonds, optionally with a linker molecule such as one to ten glycine residues.

Peptidic compounds of the invention can be obtained using any known method, including, e.g., solid phase peptide synthesis techniques, where such techniques are known to those of skill in the art. Methods for synthesizing peptides are well known in the art and have been amply described in numerous publications, including, e.g., “The Practice of Peptide Synthesis” M. Bodanszky and A. Bodanszky, eds. (1994) Springer-Verlag; and Jones, The Chemical Synthesis of Peptides (Clarendon Press, Oxford) (1994). Generally, in such methods a peptide is produced through the sequential additional of activated monomeric units to a solid phase bound growing peptide chain. Also of interest is the use of submonomers in solid phase synthesis, as described in WO 94/06451, the disclosure of which is herein incorporated by reference.

Instead of solid phase synthesis, the subject peptidic compounds of the subject invention may be prepared through expression of an expression system comprising a polynucleotide encoding the peptidic compound. Any convenient methodology may be employed, where methodologies that may be employed typically include preparation of a nucleic acid molecule comprising a nucleotide sequence encoding the subject peptide, introduction of the encoding region into a vector for expression, transformation of a host cell with the vector, and expression and recovery of the product. Protocols for accomplishing each of the above steps are well known in art. See Sambrook, Fritsch & Maniatis, Molecular Cloning, A Laboratory Manual (Cold Spring Harbor Press, Inc.) (1989).

### Therapeutic Methods

The present invention is a method for promoting angiogenesis by administering an effective amount of a peptidic compound, e.g. AC-100, which enhances angiogenesis through the induction of pro-angiogenic factors such as FGF2, TGFβ and fibronectin.

The methods of the present invention may be used to treat or prevent diseases and conditions which involve angiogenesis by administering an effective amount of the peptidic compound alone or in combination with one or more additional therapeutic agents.

Examples of conditions and diseases amenable to treatment according to the method of the invention include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangitis obliterans, thrombotic disorders, vasculitis, and the like. Examples of conditions or diseases that can be prevented using the methods of the invention include, but are not necessarily limited to, heart attack (myocardial infarction) or other vascular death, stroke, death or loss of limbs associated with decreased blood flow, and the like.

Other forms of therapeutic angiogenesis include, but are not necessarily limited to, the use of nicotine receptor agonists to accelerate healing of wounds or ulcers; to improve the vascularization of skin grafts or reattached limbs so as to preserve their function and viability; to improve the healing of surgical anastomoses (e.g., as in re-connecting portions of the bowel after gastrointestinal surgery); and to improve the growth of skin or hair.

As used herein, an “effective amount” of the peptidic compound for use with the subject methods is an amount that enhances angiogenesis by at least about 10%, at least about 15%, at least about 20%, at least about 25%, at least about 30%, at least about 35%, at least about 40%, at least about 45%, at least about 50%, at least about 55%, or at least about 60%, or more, when compared to a suitable control. Suitable controls are, in the case of experimental animals, an animal not treated with the peptide, e.g., treated with vehicle, or treated with an irrelevant peptide; and in the case of human subjects, a human subject treated with a placebo, or a human subject before treatment with a peptide of the invention.

In some embodiments, an effective amount of peptidic compound for use with the subject methods is an amount that accelerates wound healing, by at least about 10%, at least about 15%, at least about 20%, at least about 25%, at least about 30%, at least about 35%, at least about 40%, at least about 45%, at least about 50%, at least about 55%, or at least about 60%, or more, when compared to a suitable control.

In other embodiments, an effective amount of peptidic compound is an amount that increases the probability of survival of the individual suffering from a disease or condition involving angiogenesis. For example, in these embodiments, an effective amount of AC-100 is an amount effective to increase the probability of survival of an individual suffering from a disease or condition involving angiogensis by at least about 10%, at least about 15%, at least about 20%, or at least about 25%, or more, compared to the expected probability of survival without administration of a peptide compound formulation of the invention.

### Routes of Administration

Formulations of peptidic compound for use with the subject methods are administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, rectal, nasal, oral and other parenteral routes of administration. Routes of administration may be combined, if desired, or adjusted depending upon the immunomodulatory nucleic acid molecule and/or the desired effect on the immune response. The peptidic compound formulations for use with the methods of the present invention can be administered in a single dose or in multiple doses and can be provided systemically or locally.

The peptidic compound formulations can be administered to a subject using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of peptides of the invention. Where systemic delivery is desired, administration typically involves invasive or systemically absorbed topical or mucosal administration of pharmaceutical preparations.

The peptidic compound formulations of the invention can also be delivered to the subject by enteral administration. Enteral routes of administration include, but are not necessarily limited to, oral and rectal (e.g., using a suppository) delivery.

Methods of administration of the peptidic compound formulation through the skin or mucosa include, but are not necessarily limited to, topical application of a suitable pharmaceutical preparation with or without a permeation enhancer, transdermal transmission, injection and epidermal administration. Also contemplated for delivery of the peptidic compound formulation of the invention is a patch containing therein a peptide of the invention. A patch can be applied to the skin, or to other tissue, e.g., gum tissue. Any known patch delivery system that is suitable for oral delivery system can be used. See, e.g., U.S. Pat. No. 6,146,655.

Peptidic compound formulations of the present invention can also be delivered to an individual by administering to the individual a nucleic acid molecule comprising a nucleotide sequence that encodes the peptide of the invention. The terms “polynucleotide” and “nucleic acid molecule” are used interchangeably herein to refer to polymeric forms of nucleotides of any length. The polynucleotides may contain deoxyribonucleotides, ribonucleotides, and/or their analogs. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Vectors include, but are not limited to, plasmids; cosmids; viral vectors; artificial chromosomes (YAC's, BAC's, etc.); mini-chromosomes; and the like. Vectors are amply described in numerous publications well known to those in the art, including, e.g., Short Protocols in Molecular Biology, (1999) F. Ausubel, et al., eds., Wiley & Sons.

Expression vectors may be used to introduce a nucleic acid molecule encoding the AC-100 peptide into a cell of an individual. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

An expression vector comprising a nucleotide sequence encoding the peptidic compound of the invention may be introduced into tissues or host cells by any number of routes, including viral infection, microinjection, or fusion of vesicles. Jet injection may also be used for intramuscular administration, as described by Furth et al. (1992), Anal Biochem 205:365-368. The expression vector may be coated onto gold microparticles, and delivered intradermally by a particle bombardment device, or “gene gun” as described in the literature (see, for example, Tang et al. (1992), Nature 356:152-154), where gold microprojectiles are coated with the expression vector, then bombarded into skin cells.

Dosages

Although the dosage used will vary depending on the clinical goals to be achieved, a suitable dosage range is one which provides up to about 1 μg, to about 1,000 μg, to about 10,000 μg, to about 25,000 μg or about 50,000 μg of the AC-100 formulation used with the subject methods of the present invention. AC-100 formulations of the invention can be administered in a single dosage or several smaller dosages over time. Alternatively, a target dosage of a peptide can be considered to be about 0.1-1000 μM, about 1-500 μM, or about 5-250 μM in a sample of host blood drawn within the first 24-48 hours after administration of the peptide. In one embodiment the formulation is administered one time and not administered again.

The effect on angiogenesis may be dose-dependent. Therefore, to increase potency by a magnitude of two, each single dose is doubled in concentration. Increased dosages may be needed to achieve the desired therapeutic goal. The invention thus contemplates the administration of multiple doses to promote angiogenesis. When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose.

In view of the teaching provided by this disclosure, those of ordinary skill in the clinical arts will be familiar with, or can readily ascertain, suitable parameters for administration of peptides according to the invention.

### Combination Therapy

In some embodiments, a subject method of treating diseases or conditions that involve angiogenesis comprises administering the peptidic compound in an individual and at least a second therapeutic agent. Factors that control inflammation, such as ibuprofen or steroids, can be part of the pharmaceutical composition to reduce swelling and inflammation associated with the disease or condition involving angiogenesis.

Additional therapeutic agents that are suitable for use in a subject combination therapy include, but are not limited to antiinflammatory agents.

### Antiinflammatory Agents

Suitable anti-inflammatory agents include, but are not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone, benzbromarone, betamethasone, glucocorticoidspropionic acid derivatives, alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, zomepirac, flufenamic acid, meclofenamic acid, mefenamic acid, niflumic acid, olfenamic acid, diflunisal, ufenisal, isoxicam, piroxicam, sudoxicam, tenoxican, acetyl salicylic acid, sulfasalazine, apazone, bezpiperylon, feprazone, mofebutazone, oxyphenbutazone, phenylbutazone, celecoxib and rofecoxib

Formulations

In general, the peptidic compound formulations are prepared in a pharmaceutically acceptable composition for delivery to a host. Pharmaceutically acceptable carriers preferred for use with the peptides of the invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/ aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising a peptide of the invention may also be lyophilized using means well known in the art, for subsequent reconstitution and use according to the invention. Also of interest are formulations for liposomal delivery and formulations comprising microencapsulated peptides.

In general, the pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, antimicrobials, antioxidants, chelating agents, and inert gases and the like.

Absorption promoters, detergents and chemical irritants (e.g., keratinolytic agents) can enhance transmission of the peptidic formulation into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of the peptidic compound to specific tissue. Colloidal dispersion systems include macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. It has been shown that large unilamellar vesicles (LUV), which range in size from 0.2-4.0 Fm can encapsulate a substantial percentage of an aqueous buffer comprising large macromolecules. RNA and DNA can be encapsulated within the aqueous interior and be delivered to cells in a biologically active form (Fraley, et al., (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Exemplary liposome/therapeutic nucleic acid compositions suitable for use in a subject method are described in Louria-Hayon et al. (2002) Vaccine 20:3342.

Where desired, targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., (1988) Nuc. Acids Symp. Ser., 19:189; Grabarek, et al., (1990) Anal. Biochem., 185:131; Staros, et al., (1986) Anal. Biochem. 156:220 and Boujrad, et al., (1993) Proc. Natl. Acad. Sci. USA, 90:5728). Targeted delivery of a therapeutic peptidic formulation can also be achieved by conjugation of the therapeutic formulation to a surface of viral and non-viral recombinant expression vectors, to an antigen or other ligand, to a monoclonal antibody or to any molecule which has the desired binding specificity.

Induction of Angiogenesis in Vivo

In order to accomplish stimulation of angiogenesis in vivo (e.g., as in the context of therapeutic angiogenesis), a peptidic compound formulation can be administered in any suitable manner, preferably with pharmaceutically acceptable carriers. One skilled in the art will readily appreciate that the a variety of suitable methods of administering a formulation in the context of the present invention to a subject are available, and, although more than one route can be used to administer a particular compound, a particular route can provide a more immediate, more effective, and/or associated with fewer side effects than another route. In general, a peptidic compound formulation can be administered according to the method of the invention by, for example, a parenteral, intravenous, intra-arterial, inter-pericardial, intramuscular, intraperitoneal, transdermal, transcutaneous, subdermal, intradermal, or intrapulmonary route.

The peptidic compound may be administered locally for example, direct injection (e.g., intramuscular injection) at the desired treatment site, by introduction of the peptide intravenously at a site near a desired treatment site (e.g., into a vessel or capillary that feeds a treatment site), by intra-aterial or intra-pericardial introduction, by introduction (e.g., by injection or other method of implantation) of a peptidic formulation in a biocompatible gel or capsule within or adjacent a treatment site, by injection directly into muscle or other tissue in which increased blood flow and/or increased vascularity is desired, by rectal introduction of the formulation (e.g., in the form of a suppository to, for example, facilitate vascularization of a surgically created anastomosis after resection of a piece of the bowel), etc.

In one particular application of interest, a peptidic compound of the present invention is employed in a “biobypass” method, wherein instead of performing a more invasive procedure, such as a coronary bypass operation. The peptide formulation is administered to induce growth of new blood vessels around the blocked region. In this embodiment, the formulation can be administered in the area of and/or proximate to the ischemic tissue to stimulate angiogenesis.

In some embodiments it may be desirable to deliver the formulation directly to the wall of a vessel. One exemplary method of vessel wall administration involves the use of a drug delivery catheter, particularly a drug delivery catheter comprising an inflatable balloon that can facilitate delivery to a vessel wall. Thus, in one embodiment the method of the invention comprises delivery of a formulation to a vessel wall by inflating a balloon catheter, wherein the balloon comprises a nicotine receptor agonist formulation covering a substantial portion of the balloon. A peptidic formulation is held in place against the vessel wall, promoting adsorption through the vessel wall. In one example, the catheter is a perfusion balloon catheter, which allows perfusion of blood through the catheter while holding the peptide against the vessel walls for longer adsorption times. Examples of catheters suitable for peptidic compound application include drug delivery catheters disclosed in U.S. Pat. No. 5,558,642; U.S. Pat. Nos. 5,554,119; 5,591,129; and the like.

In another embodiment of interest, the peptidic formulation is delivered in the form of a biocompatible gel, which can be implanted (e.g., by injection into or adjacent a treatment site, by extrusion into or adjacent a tissue to be treated, etc.). Gel formulations comprising the peptidic formulation can be designed to facilitate local release for a sustained period (e.g., over a period of hours, days, weeks, etc.). The gel can be injected into or near a treatment site, e.g., using a needle or other delivery device. In one embodiment, the gel is placed into or on an instrument which is inserted into the tissue and then slowly withdrawn to leave a track of gel, resulting in stimulation of angiogenesis along the path made by the instrument. This latter method of delivery may be particularly desirable for the purpose of directing course of the biobypass.

In other embodiments it may be desirable to deliver the formulation topically, e.g., for localized delivery, e.g., to facilitate wound healing. Topical application can be accomplished by use of a biocompatible gel, which may be provided in the form of a patch, or by use of a cream, foam, and the like. Several gels, patches, creams, foams, and the like appropriate for application to wounds can be modified for delivery of formulations according to the invention (see, e.g., U.S. Pat. Nos. 5,853,749; 5,844,013; 5,804,213; 5,770,229; and the like). In general, topical administration is accomplished using a carrier such as a hydrophilic colloid or other material that provides a moist environment. Alternatively, for the purpose of wound healing, the peptidic compound formulation could be supplied, with or without other angiogenic agents in a gel or cream could be applied to the wound. An example of such an application would be as a sodium carboxymethylcellulose-based topical gel with a low bioburden containing the nicotine agonist and other active ingredients together with preservatives and stabilizers.

In other embodiments, the formulation is delivered locally or systemically, preferably locally, using a transdermal patch. Several transdermal patches are well known in the art for systemic delivery of nicotine to facilitate smoking cessation, and such patches may be modified to provide for delivery of an amount of the peptidic compound effective to stimulate angiogenesis according to the invention.

In other methods of delivery, the formulation can be administered using iontophoretic techniques. Methods and compositions for use in iontophoresis are well known in the art (see, e.g., U.S. Pat. Nos. 5,415,629; 5,899,876; 5,807,306; and the like).

The desirable extent of angiogenesis will depend on the particular condition or disease being treated, as well as the stability of the patient and possible side-effects. In proper doses and with suitable administration, the present invention provides for a wide range of development of blood vessels, e.g., from little development to essentially full development.

Stimulation of angiogenesis by administration of the peptidic compound can be controlled by administration of compounds that interfere with FGF2 mediated angiogenic stimulation. In this sense, the invention also provides for a means of controlling or inhibiting angiogenesis by interfering with the role of FGF2 in the angiogenic process. This may be accomplished, for example, by the administration of agents that inhibit the ability of FGF2 to mediate its effects through its receptor. Alternatively, FGF2-mediated angiogenesis can be controlled or inhibited by administration of inhibitors of processes downstream of FGF2 receptor signaling. The angiogenesis inhibitor may be administered in the same manner and dosages to mammals, such as humans, as described with respect to the peptidic compound.

The formulation of the present invention can be administered with any other known agent, eg. anti-inflammatory agent. A peptide of the invention can be administered simultaneously with (e.g., in admixture with, or in separate formulations) within about 15 minutes, about 30 minutes, about 60 minutes, about 2 hours, about 5 hours, about 10 hours, about 12 hours, about 24 hours, about 36 hours, about 4 days, about 7 days, or more, of another agent. In a particular embodiment the peptide is administered one time and not administered again.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Synthesis of D-00001, Etc.

Six different peptides were manually synthesized by the 9-fluorenylmethoxycarbonyl (Fmoc) strategy and prepared in the C-terminal amide form. The six peptides are as follows:

| | | |
|---|---|---|
| D-00001: | IPSDFEGSGYTDLQE | (SEQ ID NO:31) |
| D-00002: | DFEGSGYTDLQERGD | (SEQ ID NO:32) |
| D-00003; | YTDLQERGDNDISPF | (SEQ ID NO:33) |
| D-00004: | ERGDNDISPFSGDGQ | (SEQ ID NO:34) |
| D-00005: | NDISPFSGDGQPFKD | (SEQ ID NO:35) |
| D-00006: | TDLQERGDNDISPFSGDGQPFKD | (SEQ ID NO:30) |

(C-terminus amidated)

The peptide D-00006 is also referred to here as AC-100.

Amino acid derivatives and resins were purchased from Bachem, Inc., Torrance, Calif., and Novabiochem, La Jolla, Calif. The respective amino acids were condensed manually in a stepwise manner using 4-(2′,4′-dimethoxyphenyl-Fmoc-aminomethyl)-phenoxy resin. N-methyl pyrrolidone was used during the synthesis as a solvent. For condensation, diisopropylcarbodiimide/N-hydroxybenzotriazole was employed, and for deprotection of N^{α}-Fmoc groups, 20% piperidine in N-methyl pyrrolidone was employed. The following side chain protecting groups were used: Asn and Gln, trityl; Asp, Glu, Ser, and Thr, t-butyl; Arg, 2,2,5,7,8-pentamethylchroman-6-sulfonyl; and Lys, t-butoxycarbonyl. Resulting protected peptide resins were deprotected and cleaved from the resin using a trifluoroacetic acid-thioanisole-m-cresol-ethanedithiol-H₂O (80:5:5:5:5, v/v) at 20° C. for 2 h. Resulting crude peptides were precipitated and washed with ethyl ether then purified by reverse-phase high performance liquid chromatography (using Vydac 5C18 column and a gradient of water/acetonitrile containing 0.1% trifluoroacetic acid). All peptides were obtained with 5-20% yield (from the starting resin). Purity of the peptides was confirmed by analytical high performance liquid chromatography.

Identity of the peptides was confirmed by a Sciex API IIIE triple quadrupole ion spray mass spectrometer.

### Example 2

AC-100 enhances angiogenesis through induction of pro-angiogenic factors including FGF2, TGFbeta and Fibronectin.

We have previously reported AC-100 treatment increases PGE2, TGFbeta and Fibronectin levels in the conditioned media from the murine calvarial organ culture. Histological evaluation of the bones indicated AC-100 increased the amount of new bone formation and numbers of osteoblasts.

To investigate whether AC-100 modulates bone anabolic factors that are sequestered within the bone matrix we have analysed lysates from AC-100 treated calvariae.

Initial experiments analysed the lysates using an angiogenesis antibody array (TranSignal Mouse Angiogenesis Antibody Array, Panomics). AC-100 treated lysates showed an earlier and prolonged increase in FGF2. FGF2 was increased at 2 hr in AC-100 treated bones, by 24 hr through day 3 the FGF2 signal was the same as the control bones and at day 5 the FGF2 signal in AC-100 treated bones was still strong whereas in the control bones the signal was significantly reduced.

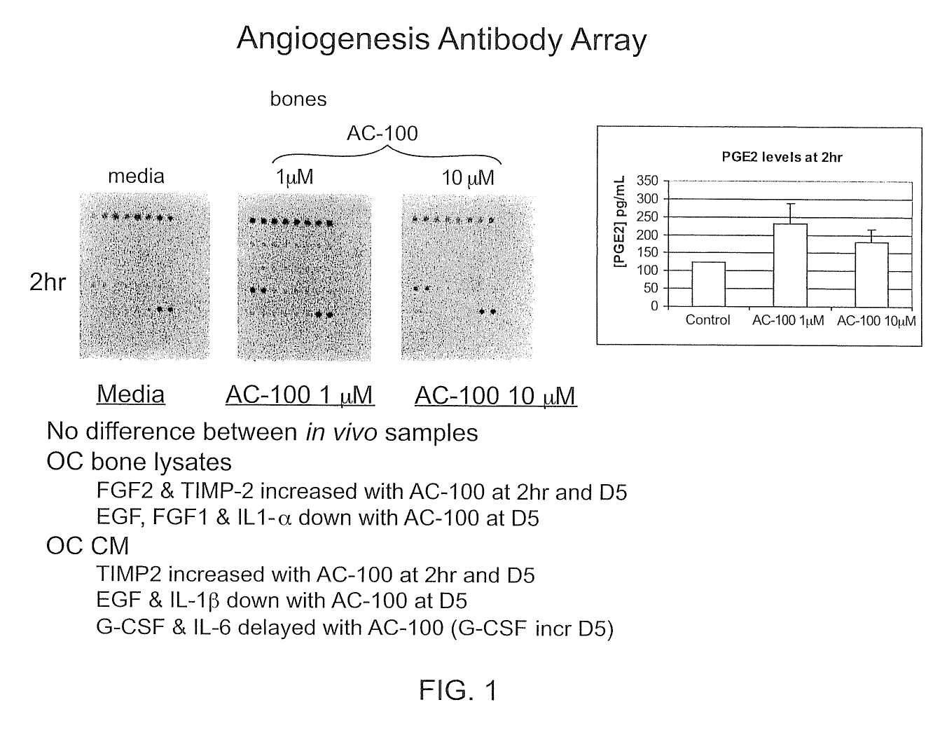

FIG. 1 depicts data demonstrating that AC-100 is able to increase the production of FGF-2 in bone. AC-100 at both 1 and 10 uM concentrations increased the production of FGF-2 in mouse calvaria organ cultures. Increases were found in both tissue lysates and in the conditioned media of cultures treated with AC-100.

The increase in FGF2 correlated with the induction of the bone anabolic factor PGE2, which we have previously reported to be a potential mechanism of action for AC-100.

To quantitatively measure the increase in FGF2 in AC-100 treated bones, lysates were harvested over a comprehensive timecourse (30 min, 1 hr, 2 hr, 6 hr, 24 hr, 48 hr, 72 hr, day 4, day 5) and analysed for FGF2 and other bone anabolic factors using ELISA. AC-100 induced a potent increase in FGF2 from the earliest timepoint through to the last time point at day 5. FGF2 was not detectable from the conditioned media suggesting AC-100 promotes FGF2 production and accumulation within the bone matrix. Consistent with our previous findings AC-100 increased PGE2 in the conditioned media and TGFbeta and Fibronectin in both the conditioned media and bone lysate.

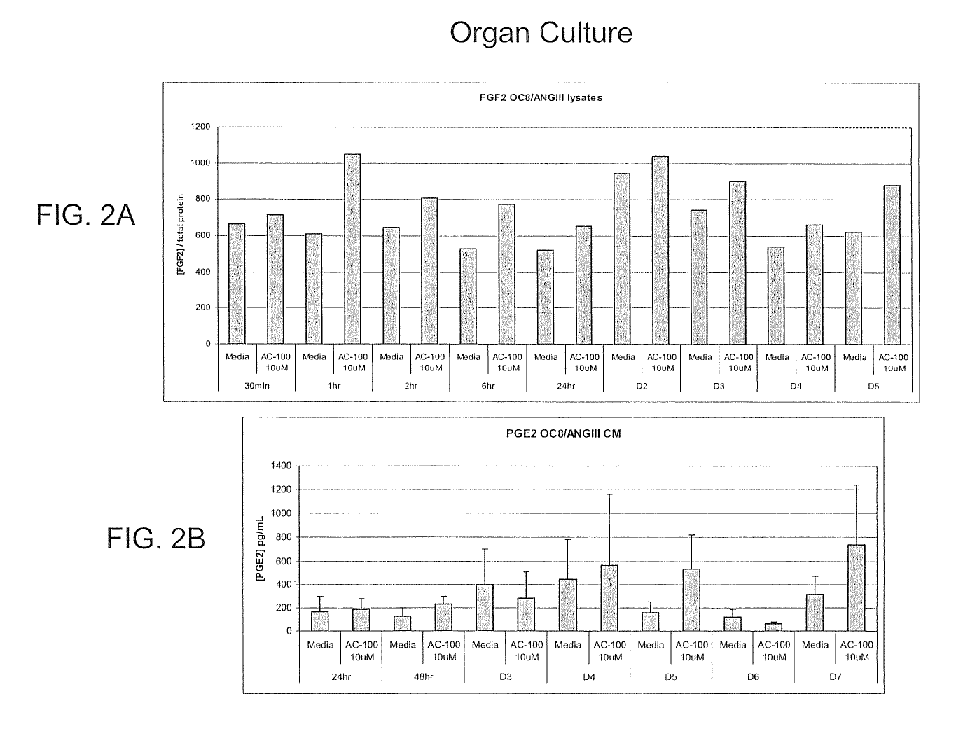

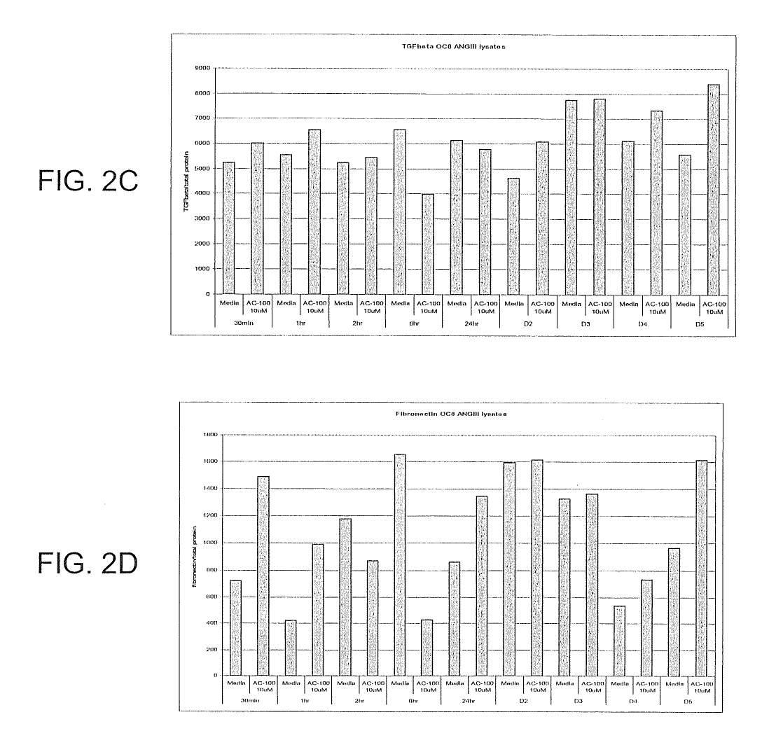

FIGS. 2A-2D depict data demonstrating that the production of FGF-2 was increased over a several day time period ranging from approximately 1 hr to 5 days (FIG. 2A). In addition, this response roughly correlated with expression of PGE-2 following AC-100 treatment (2B), TGF-β (2C) and Fibronectin (2D). Increases in PGE2 in the same cultures were observed from days 2-7.

BMP2, a potent osteogenic agent is also a strong stimulator of angiognenesis. AC-100 has shown synergistic activities with BMP2 both in vitro and in vivo. Ossicles formed with BMP2 and AC-100 co-treatment have a more mature structure than with BMP2 alone. The ossicles formed with co-treatment also contain mature osteons with new blood vessels.

Angiogenesis is a crucial part of bone healing. In diverse animal models AC-100 has consistently induced earlier healing. This earlier healing certainly contributes to the increased amount and better quality of new bone formed with AC-100 treatment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of promoting angiogenesis in a patient, comprising:
administering to a patient a formulation comprising a pharmaceutically acceptable carrier and a peptide comprising not less than 10 and not more than 30 amino acid residues, wherein the peptide comprises an integrin binding motif and a glycosaminoglycan binding motif; and
allowing the formulation to remain in contact with cells for a sufficient period of time and in sufficient amount so to stimulate angiogenesis.

2. The method of claim 1, wherein said administering is chosen from intramuscular, by inhalation, topical, transdermal, intravenous, intra-arterial, and intra-pericardial.

3. The method of claim 1, wherein said administering is directly to a local site.

4. The method of claim 1, wherein said administering is systemic.

5. The method of claim 1, wherein said administering is via a balloon catheter with formulation on a surface of the balloon which formulation is held against an internal surface of a blood vessel.

6. The method of claim 1, further comprising:
performing surgery on the patient.

7. The method of claim 6, wherein the formulation is administered locally at a site chosen from an anastomosis, suture line and a surgical wound.

8. The method of claim 1, wherein the formulation is locally administered to ischemic tissue.

9. The method of claim 1, wherein the peptide has a sequence chosen from

| | |
|---|---|
| IPSDFEGSGYTDLQE; | (SEQ ID NO:31) |
| DFEGSGYTDLQERGD; | (SEQ ID NO:32) |
| YTDLQERGDNDISPF; | (SEQ ID NO:33) |
| ERGDNDISPFSGDGQ; | (SEQ ID NO:34) |
| NDISPFSGDGQPFKD; | (SEQ ID NO:35) |
| and | |
| TDLQERGDNDISPFSGDGQPFKD. | (SEQ ID NO:30) |

10. A method for increasing blood flow to ischemic tissue proximal to a constricted blood vessel region, comprising the step of:
locally administering a therapeutically effective amount of formulation comprising a pharmaceutically acceptable carrier and a peptide comprising not less than 10 and not more than 30 amino acid residues, wherein the peptide comprises an integrin binding motif and a glycosaminoglycan binding motif.

11. The method of claim 10, wherein the formulation is administered via a catheter.

12. The method of claim 11, wherein the catheter is a balloon catheter.

13. The method of claim 10, wherein the formulation in locally administered to a blood vessel wall.

14. The method of claim 10, wherein the peptide has a sequence chosen from

| | |
|---|---|
| IPSDFEGSGYTDLQE; | (SEQ ID NO:31) |
| DFEGSGYTDLQERGD; | (SEQ ID NO:32) |
| YTDLQERGDNDISPF; | (SEQ ID NO:33) |
| ERGDNDISPFSGDGQ; | (SEQ ID NO:34) |
| NDISPFSGDGQPFKD; | (SEQ ID NO:35) |
| and | |
| TDLQERGDNDISPFSGDGQPFKD. | (SEQ ID NO:30) |

15. The method of claim 10, wherein the peptide has the sequence

| | |
|---|---|
| TDLQERGDNDISPFSGDGQPFKD. | (SEQ ID NO:30) |

16. A method of treatment, comprising:
inserting a catheter into a blood vessel;
administering from the catheter to a local site a formulation comprising a pharmaceutically acceptable carrier and a peptide consisting of the sequence

| | |
|---|---|
| TDLQERGDNDISPFSGDGQPFKD; | (SEQ ID NO: 30) |

and
allowing the formulation to promote angiogenesis.

17. The method of claim 16, wherein the formulation further comprises an anti-inflammatory compound.

18. The method of claim 16, wherein the formulation further comprises a nicotine agonist.

19. A bandage, comprising:
a structural base material;
a peptide comprising not less than 10 and not more than 30 amino acid residues, wherein the peptide comprises an integrin binding motif and a glycosaminoglycan binding motif.

20. The bandage of claim 19, wherein the base material is a carboxymethylcellulose gel and the peptide has a sequence chosen from

| | |
|---|---|
| IPSDFEGSGYTDLQE; | (SEQ ID NO:31) |
| DFEGSGYTDLQERGD; | (SEQ ID NO:32) |
| YTDLQERGDNDISPF; | (SEQ ID NO:33) |
| ERGDNDISPFSGDGQ; | (SEQ ID NO:34) |
| NDISPFSGDGQPFKD; | (SEQ ID NO:35) |
| and | |
| TDLQERGDNDISPFSGDGQPFKD. | (SEQ ID NO:30) |


SEQUENCE LISTING (35 sequences)

SEQ ID NO: 1 - protein, 13 aa, human
RGDNDISPFSGDG

SEQ ID NO: 2 - protein, 13 aa, human
RGDNDMSPFSGDG

SEQ ID NO: 3 - protein, 13 aa, human
RGDNDVPPFSGDG

SEQ ID NO: 4 - protein, 13 aa, human
RGDNDISPFSGDG

SEQ ID NO: 5 - protein, 18 aa, human
RGDNDISPFSGDGQPFKD

SEQ ID NO: 6 - protein, 18 aa, human
RGDNDMSPFSGDGQPFKD

SEQ ID NO: 7 - protein, 18 aa, human
RGDNDVPPFSGDGQHFMH

SEQ ID NO: 8 - protein, 23 aa, human
RGDNDISPFSGDGQPFKDIPGKG

SEQ ID NO: 9 - protein, 30 aa, human
RGDNDISPFSGDGQPFKDIPGKGEATGPDL

SEQ ID NO: 10 - protein, 10 aa, human
SGDGLQERGD

SEQ ID NO: 11 - protein, 10 aa, human
SGPDLLVRGD

SEQ ID NO: 12 - protein, 13 aa, human
SGDGYTDLQERGD

SEQ ID NO: 13 - protein, 12 aa, human
SGDGPDLLVRGD

SEQ ID NO: 14 - protein, 18 aa, human
IPSDFEGSGYTDLQERGD

SEQ ID NO: 15 - protein, 23 aa, human
SKVKKIPSDFEGSGYTDLQERGD

SEQ ID NO: 16 - protein, 30 aa, human
IDYLKHLSKVKKIPSDFEGSGYTDLQERGD

SEQ ID NO: 17 - protein, 14 aa, human
ERGDNDISPFSGDG

SEQ ID NO: 18 - protein, 19 aa, human
YTDLQERGDNDISPFSGDG

SEQ ID NO: 19 - protein, 24 aa, human
YTDLQERGDNDISPFSGDGQPFKD

SEQ ID NO: 20 - protein, 26 aa, human
SDFEGSGYTDLQERGDNDISPFSGDG

SEQ ID NO: 21 - protein, 17 aa, human
SGYTDLQERGDNDISPF

SEQ ID NO: 22 - protein, 18 aa, human
PDLLVRGDNDVPPFSGDG

SEQ ID NO: 23 - protein, 23 aa, human
PDLLVRGDNDVPPFSGDGQHFMH

SEQ ID NO: 24 - protein, 23 aa, human
TDLQERGDNDMSPFSGDGQPFKD

SEQ ID NO: 25 - protein, 26 aa, human
KKIPSDFEGSGYTDLQERGDNDISPF

SEQ ID NO: 26 - protein, 30 aa, human
YTDLQERGDNDISPFSGDGQPFKDIPGKGE

SEQ ID NO: 27 - protein, 30 aa, human
KKIPSDFEGSGYTDLQERGDNDISPFSGDG

SEQ ID NO: 28 - protein, 4 aa, human
SGDG

SEQ ID NO: 29 - protein, 12 aa, Artificial Sequence
  binding motif
DXDXSXFXGXXQ

SEQ ID NO: 30 - protein, 23 aa, Artificial Sequence
  synthetic peptide
TDLQERGDNDISPFSGDGQPFKD

SEQ ID NO: 31 - protein, 15 aa, Artificial Sequence
  synthetic peptide
IPSDFEGSGYTDLQE

SEQ ID NO: 32 - protein, 15 aa, Artificial Sequence
  synthetic peptide
DFEGSGYTDLQERGD

SEQ ID NO: 33 - protein, 15 aa, Artificial Sequence
  synthetic peptide
YTDLQERGDNDISPF

SEQ ID NO: 34 - protein, 15 aa, Artificial Sequence
  synthetic peptide
ERGDNDISPFSGDGQ

SEQ ID NO: 35 - protein, 15 aa, Artificial Sequence
  synthetic peptide
NDISPFSGDGQPFKD